(19) United States
(12) Patent Application Publication
(10) Pub. No.: US 2024/0366087 A1
(43) Pub. Date: Nov. 7, 2024

(54) SYSTEM AND METHOD FOR DETERMINING NETWORKS OF BRAIN FROM RESTING STATE MRI DATA USING ML

(71) Applicant: BRAINSIGHT TECHNOLOGY PRIVATE LIMITED, Bangalore (IN)

(72) Inventors: Rimjhim Agrawal, Bangalore (IN); Ruchi Sharma, Bhopal (IN); Dilip Rajeswari, Bangalore (IN); Akshay Kumaar Murali Gopika Manoharan, Chennai (IN)

(21) Appl. No.: 18/285,639

(22) PCT Filed: Apr. 5, 2022
(86) PCT No.: PCT/IN2022/050334

§ 371 Date: Oct. 4, 2023

(30) Foreign Application Priority Data
Apr. 5, 2021 (IN) 202141016062

(51) Int. Cl. A61B 5/00; A61B 5/055

(52) U.S. Cl. CPC A61B 5/0042; A61B 5/055; A61B 5/7264

(57) ABSTRACT

A method for determining networks of a brain of a subject (102) from a resting state magnetic resonance imaging (MRI) data using a machine learning model (112) for evaluating health conditions of the subject is provided. The method includes obtaining the input data from an imaging device (110). The input data includes T1 weighted MRI image, or a resting-state functional MRI image in a predefined format. The method includes converting the predefined format of the scan data into object format file. The method includes generating a four-dimensional (4D) functional connectivity file. The method includes decomposing the 4D functional connectivity file into a n-component specified time-series. The method includes providing a spatial relationship between seed-region of the brain and rest of the object format file of the scan data when combined. The method includes determining, using an Intraoperative Direct Electrical Stimulation (DES) localization method and machine learning model, networks of brain.

### TECHNICAL FIELD

The embodiments herein generally relate to the identification of networks of the brain, more particularly, a system and method for identifying networks of the brain from resting-state magnetic resonance imaging (MRI) data using a machine learning model.

### DESCRIPTION OF THE RELATED ART

Large-scale brain networks are brain regions that show functional connectivity in the brain by analyzing magnetic resonance imaging (MRI) scan, positron emission tomography (PET) scan, etc. The mapping of such networks for any patients with large tumors in the brain or any accident cases, in a pre-surgical stage in a non-invasive way, would be a challenging task. This mapping of networks provides neurosurgeons with an idea to plan further based on criticality.

Existing approaches use correlation-based analysis to derive one or more networks of the brain. The correlation-based analysis provides one or more networks of the brain based on a correlation between a template and a single subject. This confines a limit to obtaining a few networks in a particular region of interest.

Existing systems employ convolutional neural networks (CNN) to model activation time courses, decomposition techniques, seed-based analysis, and multi-layer perceptron (MLP) to derive networks of the brain. The existing systems fail to provide a wide collection of networks of the brain precisely. The existing systems fail to assess the wide collection of networks of the brain to provide an idea about the condition of the patient easily.

Therefore, there arises a need to address the aforementioned technical drawbacks in existing technologies to identify networks of the brain.

### SUMMARY

In view of the foregoing, an embodiment herein provides a system for determining networks of a brain of a subject from a resting state magnetic resonance imaging (MRI) data using a machine learning model for evaluating health conditions of the subject. The system includes an imaging device. The imaging device includes at least one of a camera or a screen. The imaging device obtains an input data of the subject that includes at least one of scan data. The scan data includes at least one of T1 weighted magnetic resonance imaging (MRI) image, or a resting-state functional MRI image in a predefined format. The system includes a brain network identifying server that acquires the input data of the subject from the imaging device, and processes, the input data using the machine learning model. The brain network identifying server includes a memory that stores a database, a processor that is configured to execute the machine learning model and is configured to (i) convert the predefined format of the scan data into an object format file by pre-processing the predefined format of the scan data of the subject; (ii) generate, using an independent component analysis method, a four-dimensional (4D) functional connectivity file from the object format file, the 4D functional connectivity file includes at least one functional connectivity features; (iii) decompose the 4D functional connectivity file into a n-component specified time-series, the n-component specified time-series comprises time components that are independent of each other statistically; (iv) train, using data analysis pipelines, the machine learning model by providing historical input data of historical subjects and historical brain networks associated with the historical subjects as training data to obtain a trained machine learning model; (v) obtain, using a multi-seed-based correlation analysis, networks of the brain of the subject by providing a spatial relationship between a seed-region of the brain of the subject and rest of the object format file of the scan data of the subject when combined; (vi) compose the networks of the brain of the subject by assigning a defined threshold for a set of voxels of a set of seed-regions of the brain of the subject; and (vii) determine, using an Intraoperative Direct Electrical Stimulation (DES) localization method and the trained machine learning model, the networks of the brain by comparing the composed networks of the brain with a template that defines a network of interest, the brain network identifying server enables the evaluation of health conditions of the subject using the networks that are determined.

In some embodiments, the processor is configured to pre-process the dicom format of the input data of the subject to obtain a pre-processed dicom format input file by, (i) discarding a first ten functional time-series volumes of the input data of the subject; (ii) interpolating slices of the input data of the subject by acquiring the input data at a single time point by compensating time differences between slice acquisitions of the input data of the subject; (iii) correcting a head-movement of the subject that usually occurs during an acquisition of the input data along at least one of X, Y, or Z movement axes and at least one of X, Y, and Z rotation axes; (iv) removing linear or quadratic trends in the functional time-series volumes of the input data of the subject; (v) registering the input data by aligning a functional image of the input data with the reference to a structural image in the input data of the subject; (vi) stripping the head of the subject to improve the robustness of the registration to the input data using Montreal Neurological Institute (MNI) normalization; (vii) segmenting the registered input data into a grey matter, a white matter, and a cerebrospinal fluid; (viii) generating a mask for the segmented input data of the brain and scalp regions in both the subject's native space and group mask; (ix) removing noise in a signal induced by the head-movement using regressors, scanner drift using a linear term, and global functional MRI signals from the white matter and the cerebrospinal fluid segments; (x) filtering noise due to low-frequency drifts and physiological noise using a high-pass filter to remove drifts in neighbouring voxels of the input data; and (xi) removing a small scale that changes among voxels due to an increase in the signal-to-noise ratio of the input data by filtering the high range frequencies from frequency domain.

In some embodiments, the processor is configured to obtain stable networks of the brain by implying a multi-seed-based correlation analysis by (i) combining the networks from the multiple seed-regions of the brain of the subject within a selected region of interest (ROI) and (ii) weighing the multiple seed-region of the brain of the subject based on a distance from the main seed-region of the brain of the subject.

In some embodiments, the processor is configured to incorporate a dynamic thresholding to increase the threshold of the plurality of networks of the brain to assess an overlap, if the overlap is within an optimal bound, then an optimal thresholding is applied, composed networks of the brain is converted into at least one of the predefined format, or the object format file after optimal thresholding.

Intraoperative Direct Electrical Stimulation (DES) localization is used for correlation to enable better seed-selection and direct comparison between the DES activation and n brain networks.

In some embodiments, the processor is configured to validate the networks of the brain by comparing the networks of the brain that are obtained based on tasks performed by the subject and correlating with the networks of the brain while the subject is performing a specified task using the interoperative DES localization method, the networks of the brain obtained from task-based are the plurality of networks of the brain obtained from task-based.

In some embodiments, the interoperative DES localization method performs correlation to enable selection of the seed-region of the brain and comparison between the Intraoperative Direct Electrical Stimulation activation and the networks of the brain.

In some embodiments, the networks of the brain is at least one of a primary visual network and sensorimotor network of the brain, a language network, a dorsal default mode network of the brain, a posterior salience network, or a right executive control network of the brain.

In some embodiments, the defined threshold is assigned using a statistical significance for the set of voxels given by the time components of the n-component specified time-series.

In one aspect, a processor-implemented method for determining networks of a brain of a subject from a resting state magnetic resonance imaging (MRI) data using a machine learning model for evaluating health conditions of the subject is provided. The method includes obtaining the input data of a subject from an imaging device that includes at least one of a camera, or a screen. In some embodiments, the input data includes at least one of at least one scan data. In some embodiments, the scan data includes at least one T1 weighted magnetic resonance imaging (MRI) image, or a resting-state functional MRI image in a predefined format. The method includes acquiring the input data of the subject from the imaging device, and processing the input data using the machine learning model. The method includes converting the predefined format of the scan data into an object format file by pre-processing the predefined format of the scan data of the subject. The method includes generating, using an independent component analysis method, a four-dimensional (4D) functional connectivity file from the object format file, the 4D functional connectivity file includes at least one functional connectivity features. The method includes decomposing the 4D functional connectivity file into a n-component specified time-series, the n-component specified time-series comprises time components that are independent of each other statistically. The method includes training, using data analysis pipelines, the machine learning model by providing historical input data of historical subjects and historical brain networks associated with the historical subjects as training data to obtain a trained machine learning model. The method includes obtaining, using a multi-seed-based correlation analysis, networks of the brain of the subject by providing a spatial relationship between a seed-region of the brain of the subject and rest of the object format file of the scan data of the subject when combined. The method includes composing the networks of the brain of the subject by assigning a defined threshold for a set of voxels of a set of seed-regions of the brain of the subject. The method includes determining, using an Intraoperative Direct Electrical Stimulation (DES) localization method and the trained machine learning model, the networks of the brain by comparing the composed networks of the brain with a template that defines a network of interest, the brain network identifying server enables the evaluation of health conditions of the subject using the networks that are determined.

The system and/or method is used for deriving a wide range of networks of the brain. Using the wide range of networks of the brain, the system assesses brain tumors like glioma, meningioma, epilepsy, traumatic brain injury, etc. The wide range of networks of the brain is eloquent cortex mapping (ECM) networks like motor networks, visual networks, etc, resting-state networks (RSN), default mode networks (DMN) networks, frontoparietal networks, etc.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 is a block diagram of a system for determining networks of a brain of a subject from a resting state magnetic resonance imaging (MRI) data using a machine learning model for evaluating health conditions of the subject according to some embodiments herein;

FIG. 2 is a block diagram of a brain network determining server of FIG. 1 according to some embodiments herein;

FIGS. 3A-3C are exemplary views of one or more networks of the brain of FIG. 1 according to some embodiments herein;

FIGS. 4A-4B illustrate a flow diagram of a method for determining networks of a brain of a subject from a resting state magnetic resonance imaging (MRI) data using a machine learning model for evaluating health conditions of the subject according to some embodiments herein; and

FIG. 5 is a schematic diagram of a computer architecture in accordance with the embodiments herein.

### DETAILED DESCRIPTION OF THE DRAWINGS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

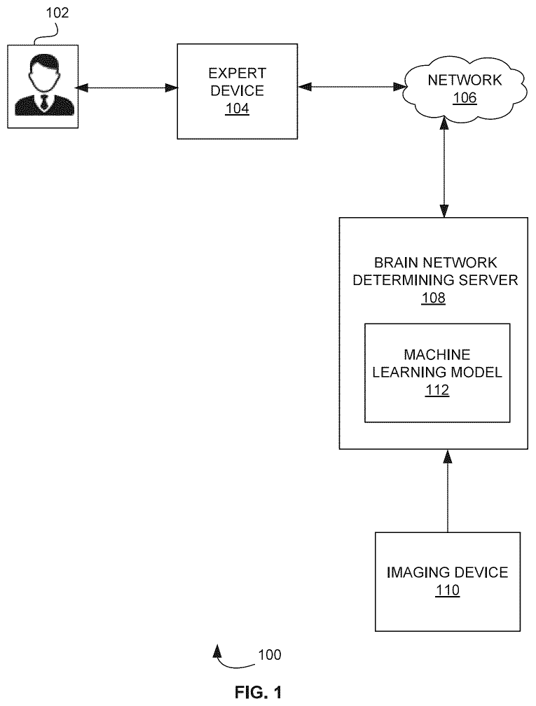

As mentioned, there is a need for a system to determine networks of a brain of a subject from a resting state magnetic resonance imaging (MRI) data using a machine learning model for evaluating the health conditions of the subject. Referring now to the drawings, and more particularly to FIGS. 1 through 5, where similar reference characters denote corresponding features consistently throughout the figures, preferred embodiments are shown.

FIG. 1 is a block diagram of a system 100 for determining networks of a brain of a subject 102 from a resting state magnetic resonance imaging (MRI) data using a machine learning model 112 for evaluating health conditions of the subject 102 according to some embodiments herein. The system 100 includes an expert device 104, a network 106, a brain network determining server 108, an imaging device 110, and a machine learning model 112. In some embodiments, the system 100 includes an android application package (APK), iOS App Store Package (IPA), or any such application packages that are installed in the expert device 104 of the subject 102. In some embodiments, the expert device 104, without limitation, is selected from a mobile phone, a Personal Digital Assistant (PDA), a tablet, a desktop computer, or a laptop computer. In some embodiments, the system 100 includes an application that may be installed in android-based devices, windows-based devices, or any such mobile operating systems devices.

The expert device 104 obtains input data of the subject 102 and communicates to the brain network determining server 108 through the network 106. In some embodiments, the network 106 without limitation, is selected from a wired network or a wireless network such as Bluetooth, Wi-Fi, ZigBee, cloud, or any other communication networks. The imaging device 110 includes at least one camera or a screen. The imaging device 110 obtains the input data of the subject 102. The input data includes at least one scan data and one or more attributes.

In some embodiments, the scan data includes at least one of the input data including T1 weighted magnetic resonance imaging (MRI) scan, a resting-state functional MRI scan. In some embodiments, the input data of the subject 102 is in Dicom format or nifty format or nearly raw raster data (NRRD) or other imaging or neuroimaging formats. The input data may include clinical data of the subject 102 and demographic data of the subject 102. The clinical data may include present or prior disease (comorbidities), relapse, rehabilitation, number of days the patients survived after surgery, frequency of chemotherapy/radiotherapy, post and pre-surgery drug/medication regime, dietary changes/restrictions, blood parameters, surgical procedure and extent of surgery, rating for the functional status of the patient before and after surgery. The demographic data of the subject 102 may include user age, gender, marital status, family size, ethnicity, income, and education. The brain network determining server 108 converts the predefined format of the scan data into an object format file by pre-processing the predefined format of the scan data of the subject 102.

The predefined format of the scan data may be Dicom. The object format file may be NIfTI format. The object format file generates the corresponding header information as a JSON sidecar. The brain network determining server 108 generates a four-dimensional (4D) functional connectivity file from the object format file using an independent component analysis method. In some embodiments, the independent component analysis method is used to separate independent sources from a mixed-signal. The independent source may be the functional connectivity and the mixed-signal may be the object format file.

The brain network determining server 108 decomposes the four-dimensional connectivity file into an n-component specified time series. The n-component specified time series includes time components that are independent of each other statistically.

The machine learning model 112 is trained by providing historical input data of historical subjects and historical brain networks associated with the historical subjects as training data to obtain a trained machine learning model using one or more data analysis pipelines.

The brain network determining server 108 obtains one or more networks of the brain of the subject 102 by providing a spatial relationship between a seed-region of the subject 102 and the object format file of the scan data of the subject 102 when combined. The seed-region of the subject 102 includes at least one voxel coordinate in the input data. In some embodiments, the voxel coordinate in the input data refers to the seed-region. The voxel co-ordinate may define a position, color, and density of the image. In some embodiments, the rest of the object format file of the scan data of the subject 102 is obtained by excluding the seed-region of the brain of the subject 102 from the object format file of the scan data.

In some embodiments, the processor is configured to obtain a plurality of stable networks of the brain by implying a multi-seed based correlation analysis by (i) combining the one or more networks from the multiple seed-regions of the brain of the subject 102 within a selected region of interest (ROI) and (ii) weighing the multiple seed-region of the brain of the subject (102) based on a distance from the main seed-region of the brain of the subject 102. The multi-seed-based correlation analysis (multi-SCA) is used to explore functional connectivity within the brain. Based on the time series of multi-seed voxels or regions of interest (ROI), connectivity is calculated as the correlation of the time series for all other voxels in the brain. The result of multi-SCA is connectivity maps showing Z-scores for each voxel indicating how well the time series of the Z-scores correlates with the time series of the multi-seeds.

The brain network determining server 108 composes the one or more networks of the brain of the subject 102 by assigning a defined threshold for a set of voxels of a set of seed-regions of the brain of the subject 102. In some embodiments, the defined threshold is assigned using a statistical significance for the set of voxels given by the time components of the n-component specified time-series. In some embodiments, the brain network determining server 108 incorporates a dynamic thresholding to increase the threshold of the networks of the brain to assess an overlap. To assess the overlap, two matches provide the most overlap accuracy metric. The accuracy metric may be Sorensen-Dice coefficient or smooth Dice coefficient, etc. In some embodiments, if any region in the n brain networks overlaps with the template, and if the region of overlap is within a certain optimal bound, optimal thresholding may be applied. In some embodiments, composed networks of the brain is converted into at least one of digital imaging and communications in medicine (dicom) format or neuroimaging informatics technology initiative (NIfTI) format in both standard and native space for the subject after optimal thresholding.

The optimal bound may be a range of values of Sorensen-Dice coefficient or smooth Dice coefficient. Thresholding is a method used to estimate threshold values for segmenting the input data into distinct regions. The thresholding simplifies the representation of the input data into a threshold image that is easier to analyze and is more meaningful. The optimal thresholding is used to devise a function that yields a measure of separation between overlapping regions. The function is calculated for each intensity which maximizes the function and is chosen as the threshold.

The brain network determining server 108 determines the one or more networks of the brain of the subject 102 by comparing the composed networks of the brain with a template that defines a network of interest using an Intraoperative Direct Electrical Stimulation (DES) localization method and the trained machine learning model. In some embodiments, the brain network identifying server 108 enables the evaluation of one or more health conditions of the subject 102 using the one or more networks of the brain of the subject 102 that are determined. The Intraoperative Direct Electrical Stimulation (DES) localization method is used for mapping cognitive functions to monitor epileptogenic tissue of the brain. The electrical stimulation mapping allows for the determination of the functional cortex and helps to localize the epileptic network and the functional impact of the epileptic network.

In some embodiments, the brain network determining server 108 obtains stable networks of the brain by implying a multi-seed-based correlation analysis by (i) combining the networks from the multiple seed-regions of the brain of the subject 102 within a selected region of interest (ROI) and (ii) weighing the multiple seed-region of the brain of the subject 102 based on a distance from the main seed-region of the brain of the subject 102.

In some embodiments, the brain network determining server 108 validates the networks of the brain by comparing the networks of the brain that are obtained based on tasks performed by the subject 102 and correlating with the networks of the brain while the subject 102 is performing a specified task using the interoperative DES localization method. For example, if the task is motor-based, then the task-based n brain networks are compared to the motor network derived from the n brain networks. In some embodiments, Intraoperative Direct Electrical Stimulation (DES) localization is used for correlation to enable better seed-selection and direct comparison between the DES activation and n brain networks.

In some embodiments, the interoperative DES localization method performs correlation to enable selection of the seed-region of the brain and comparison between the Intraoperative Direct Electrical Stimulation activation and the networks of the brain.

In some embodiments, the networks of the brain are at least one of a primary visual network and sensorimotor network of the brain, a language network, a dorsal default mode network of the brain, a posterior salience network, or a right executive control network of the brain.

In some embodiments, the defined threshold is assigned using a statistical significance for the set of voxels given by the time components of the n-component specified time series.

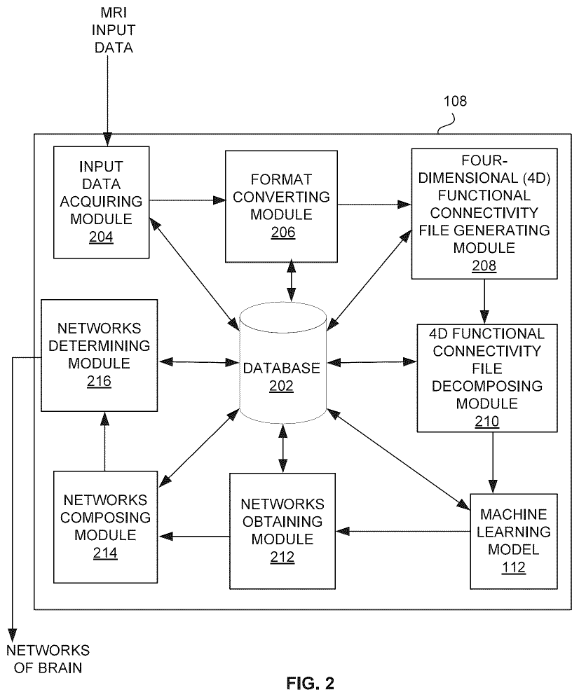

FIG. 2 is a block diagram of a brain network determining server 108 of FIG. 1 according to some embodiments herein. The brain network determining server 108 includes a database 202, an input data acquiring module 204, a format converting module 206, a four-dimensional (4D) functional connectivity file generating module 208, a 4D functional connectivity file decomposing module 210, a networks obtaining module 212, a networks composing module 214, a networks determining module 216 and the machine learning model 112.

The input data acquiring module 204 acquires the input data of the subject 102. The input data includes the scan data includes at least one of the input data includes T1 weighted magnetic resonance imaging (MRI) scan, a resting-state functional MRI scan. In some embodiments, the input data of the subject 102 is in Dicom format or nifty format or nearly raw raster data (NRRD) or other imaging or neuroimaging formats. The input data may include clinical data of the subject 102 and demographic data of the subject 102. The clinical data may include present or prior disease (comorbidities), relapse, rehabilitation, number of days the patients survived after surgery, frequency of chemotherapy/radiotherapy, post and pre-surgery drug/medication regime, dietary changes/restrictions, blood parameters, surgical procedure and extent of surgery, rating for the functional status of the patient before and after surgery. The demographic data of the subject 102 may include user age, gender, marital status, family size, ethnicity, income, and education.

The format converting module 206 converts the predefined format of the scan data into an object format file by pre-processing the predefined format of the scan data of the subject 102. The predefined format of the scan data may be Dicom. The object format file may be NIfTI format. The object format file generates the corresponding header information as a JSON sidecar.

The format converting module 206 pre-processes the predefined format of the scan data by, (i) discarding a first ten functional time-series volumes of the input data of the subject 102; (ii) interpolating slices of the input data of the subject 102 by acquiring the input data at a single time point by compensating time differences between slice acquisitions of the input data of the subject 102; (iii) correcting a head-movement of the subject 102 that usually occurs during an acquisition of the input data along at least one of X, Y, or Z movement axes and at least one of X, Y, and Z rotation axes; (iv) removing linear or quadratic trends in the functional time-series volumes of the input data of the subject 102; (v) registering the input data by aligning a functional image of the input data with the reference to a structural image in the input data of the subject 102; (vi) stripping the head of the subject 102 to improve the robustness of the registration to the input data using Montreal Neurological Institute (MNI) normalization; (vii) segmenting the registered input data into a grey matter, a white matter, and a cerebrospinal fluid; (viii) generating a mask for the segmented input data of the brain and scalp regions in both the subject's native space and group mask; (ix) removing noise in a signal induced by the head-movement using regressors, scanner drift using a linear term, and global functional MRI signals from the white matter and the cerebrospinal fluid segments; (x) filtering noise due to low-frequency drifts and physiological noise using a high-pass filter to remove drifts in neighbouring voxels of the input data; and (xi) removing a small scale that changes among voxels due to an increase in the signal-to-noise ratio of the input data by filtering the high range frequencies from frequency domain.

The 4D functional connectivity file generating module 208 generates a four-dimensional (4D) functional connectivity file from the object format file using an independent component analysis method. In some embodiments, the independent component analysis method is used to separate independent sources from a mixed-signal. The independent source may be the functional connectivity and the mixed-signal may be the object format file.

The 4D functional connectivity file decomposing module 210 decomposes the four-dimensional connectivity file into an n-component specified time series.

The machine learning model 112 is trained by providing historical input data of historical subjects and historical brain networks associated with the historical subjects as training data to obtain a trained machine learning model using one or more data analysis pipelines. In some embodiments, the machine learning model 112 may be a supervised and/or unsupervised learning model. In some embodiments, the machine learning model 112 learns about a specific brain illness and a consistent deviation of the n brain networks. The machine learning model 112 learns by obtaining one or more input data to improve the accuracy of composing the n brain networks.

The networks obtaining module 212 obtains one or more networks of the brain of the subject 102 by providing a spatial relationship between a seed-region of the subject 102 and the object format file of the scan data of the subject 102 when combined. In some embodiments, the rest of the object format file of the scan data of the subject 102 is obtained by excluding the seed-region of the brain of the subject 102 from the object format file of the scan data.

The networks composing module 214 composes the one or more networks of the brain of the subject 102 by assigning a defined threshold for a set of voxels of a set of seed-regions of the brain of the subject 102. In some embodiments, the defined threshold is assigned using a statistical significance for the set of voxels given by the time components of the n-component specified time series. In some embodiments, composed networks of the brain is converted into at least one of digital imaging and communications in medicine (dicom) format or neuroimaging informatics technology initiative (NIfTI) format in both standard and native space for the subject after optimal thresholding.

The networks determining module 216 determines the one or more networks of the brain of the subject 102 by comparing the composed networks of the brain with a template that defines a network of interest using an Intraoperative Direct Electrical Stimulation (DES) localization method and the trained machine learning model. In some embodiments, the brain network identifying server 108 enables the evaluation of one or more health conditions of the subject 102 using the one or more networks of the brain of the subject 102 that are determined.

For example, if the task is motor-based, then the task-based n brain networks are compared to the motor network derived from the n brain networks. In some embodiments, Intraoperative Direct Electrical Stimulation (DES) localization is used for correlation to enable better seed-selection and direct comparison between the DES activation and n brain networks.

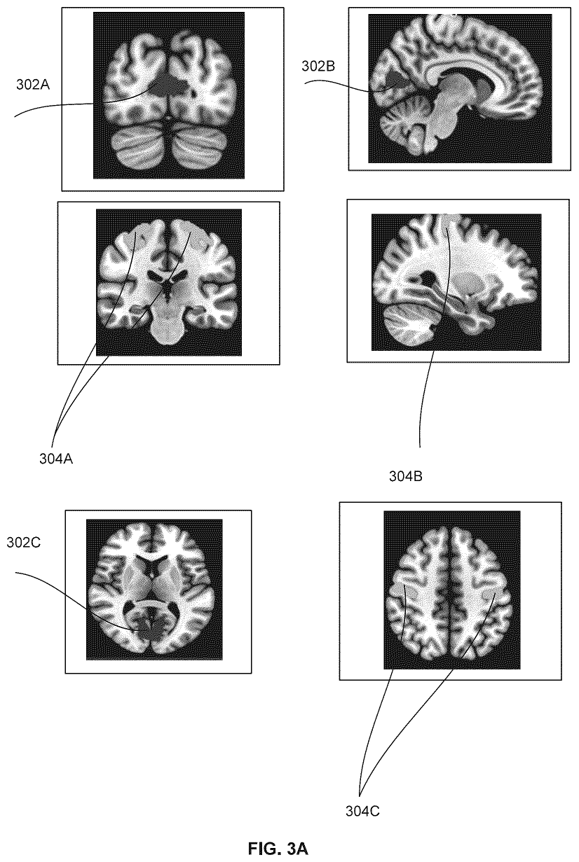

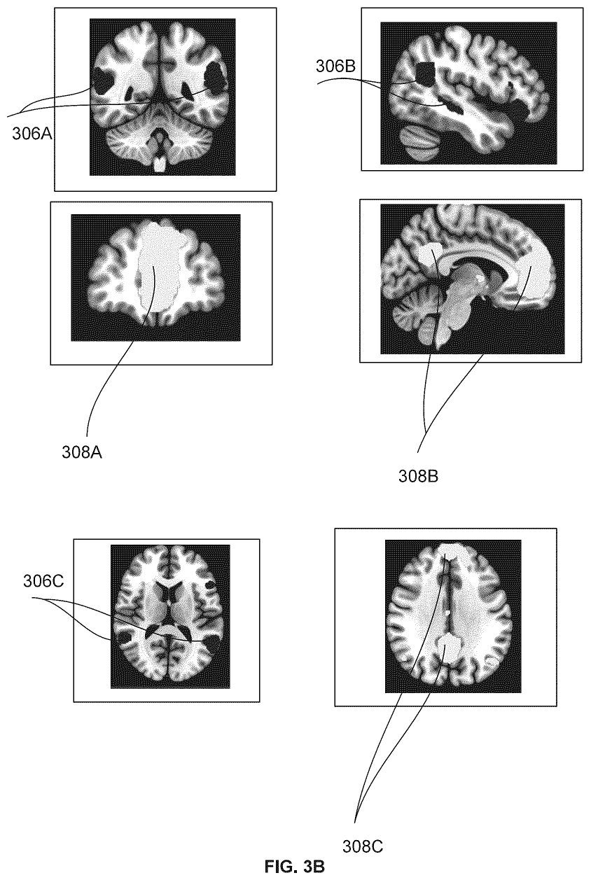

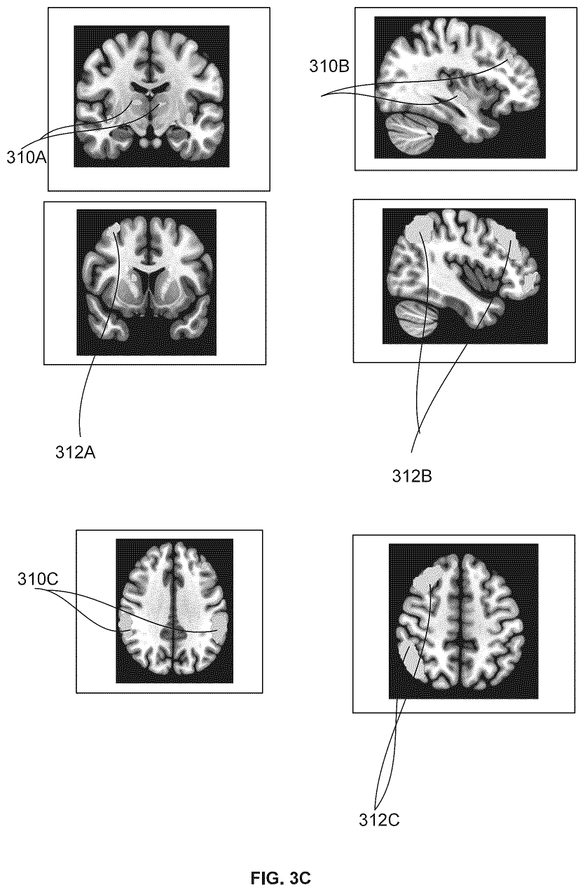

FIGS. 3A-3C are exemplary views of one or more identified networks of the brain of FIG. 1 according to some embodiments herein. FIG. 3A is an exemplary view of the primary visual network and sensorimotor network of the brain. The exemplary views at 302A, 302B, and 302C depict the primary visual network of the brain. The primary network of the brain is derived from an eloquent cortical network of the brain. The eloquent cortex refers to an area of a cortex that is disrupted or resected which may result in disruption and/or loss in language, and/or motor and/or sensory processing abilities of the brain.

The exemplary views at 304A, 304B, and 304C depict the sensorimotor network of the brain. The sensorimotor network of the brain is derived from an eloquent cortical network of the brain. The eloquent cortex refers to an area of the cortex that is disrupted or resected which may result in disruption and/or loss in language, and/or motor and/or sensory processing abilities of the brain.

FIG. 3B is an exemplary view of a language network and dorsal default mode network of the brain. The exemplary views at 306A, 306B, and 306C depict the language network of the brain. The language network of the brain is derived from an eloquent cortical network of the brain. The eloquent cortex refers to an area of the cortex that is disrupted or resected which may result in disruption and/or loss in language, and/or motor and/or sensory processing abilities of the brain.

The exemplary views at 308A, 308B, and 308C depict the dorsal default mode network of the brain. The dorsal default mode network derives from resting-state functional MRI of the brain. The dorsal default mode network allows for assessing the default functioning of the brain and also a deviation of the network.

FIG. 3C is an exemplary view of a posterior salience network and the right executive control network of the brain. The exemplary views at 310A, 310B, and 310C depict the posterior salience network of the brain. The posterior salience network derives from resting-state functional MRI of the brain. The posterior salience network allows for assessing the default functioning of the brain and also a deviation of the network.

The exemplary views at 312A, 312B, and 312C depict the right executive control network of the brain. The right executive control network derives from a resting-state functional MRI of the brain. The right executive control network allows for assessing the default functioning of the brain and also a deviation of the network.

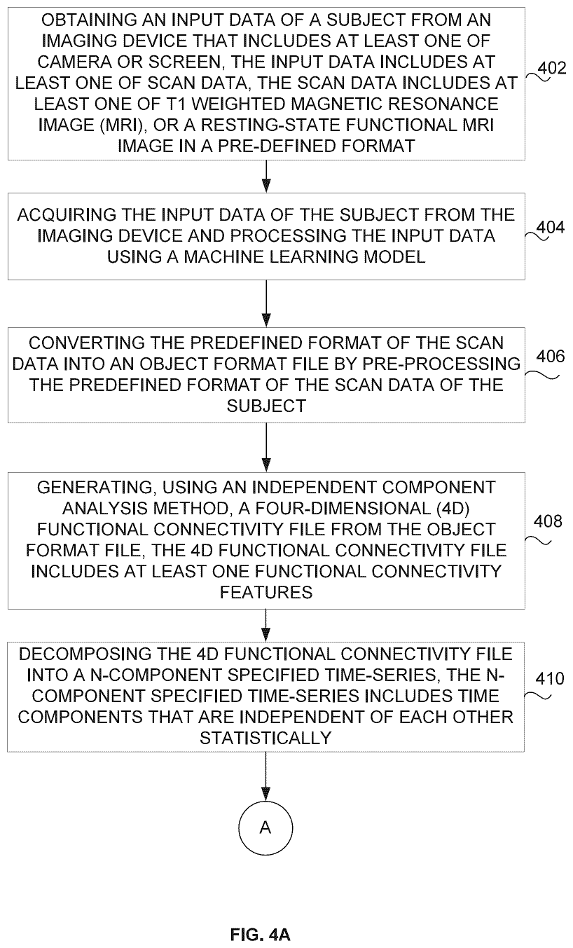

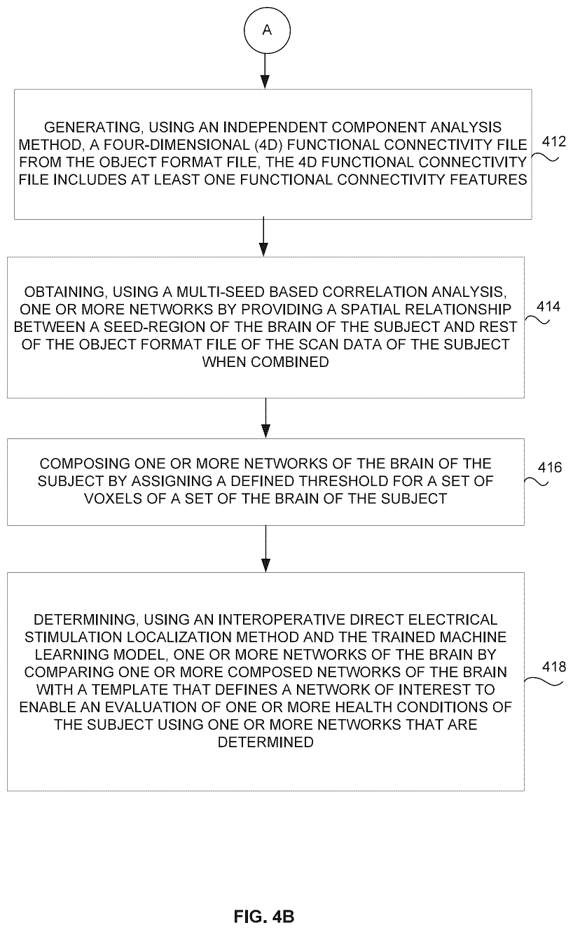

FIGS. 4A-4B illustrate a flow diagram of a method for determining networks of a brain of a subject 102 from a resting state magnetic resonance imaging (MRI) data using a machine learning model 112 for evaluating health conditions of the subject 102 according to some embodiments herein. At step 402, the method includes obtaining the input data of a subject from an imaging device that includes at least one of camera, or a screen. In some embodiments, the input data includes at least one of at least one scan data. In some embodiments, the scan data includes at least one of T1 weighted magnetic resonance imaging (MRI) image, or a resting-state functional MRI image in a predefined format. At step 404, the method includes acquiring the input data of the subject from the imaging device, and processing the input data using the machine learning model. At step 406, the method includes converting the predefined format of the scan data into an object format file by pre-processing the predefined format of the scan data of the subject. At step 408, the method includes generating, using an independent component analysis method, a four-dimensional (4D) functional connectivity file from the object format file, the 4D functional connectivity file includes at least one functional connectivity features. At step 410, the method includes decomposing the 4D functional connectivity file into a n-component specified time-series, the n-component specified time-series comprises time components that are independent of each other statistically. At step 412, the method includes training, using data analysis pipelines, the machine learning model by providing historical input data of historical subjects and historical brain networks associated with the historical subjects as training data to obtain a trained machine learning model. At step 414, the method includes obtaining, using a multi-seed-based correlation analysis, networks of the brain of the subject by providing a spatial relationship between a seed-region of the brain of the subject and rest of the object format file of the scan data of the subject when combined. At step 416, the method includes composing the networks of the brain of the subject by assigning a defined threshold for a set of voxels of a set of seed-regions of the brain of the subject. At step 418, the method includes determining, using an Intraoperative Direct Electrical Stimulation (DES) localization method and the trained machine learning model, the networks of the brain by comparing the composed networks of the brain with a template that defines a network of interest, the brain network identifying server enables the evaluation of health conditions of the subject using the networks that are determined.

In some embodiments, the processor is configured to pre-process the dicom format of the input data of the subject to obtain a pre-processed dicom format input file by, (i) discarding a first ten functional time-series volumes of the input data of the subject; (ii) interpolating slices of the input data of the subject by acquiring the input data at a single time point by compensating time differences between slice acquisitions of the input data of the subject; (iii) correcting a head-movement of the subject that usually occurs during an acquisition of the input data along at least one of X, Y, or Z movement axes and at least one of X, Y, and Z rotation axes; (iv) removing linear or quadratic trends in the functional time-series volumes of the input data of the subject; (v) registering the input data by aligning a functional image of the input data with the reference to a structural image in the input data of the subject; (vi) stripping the head of the subject to improve the robustness of the registration to the input data using Montreal Neurological Institute (MNI) normalization; (vii) segmenting the registered input data into a grey matter, a white matter, and a cerebrospinal fluid; (viii) generating a mask for the segmented input data of the brain and scalp regions in both the subject's native space and group mask; (ix) removing noise in a signal induced by the head-movement using regressors, scanner drift using a linear term, and global functional MRI signals from the white matter and the cerebrospinal fluid segments; (x) filtering noise due to low-frequency drifts and physiological noise using a high-pass filter to remove drifts in neighbouring voxels of the input data; and (xi) removing a small scale that changes among voxels due to an increase in the signal-to-noise ratio of the input data by filtering the high range frequencies from frequency domain.

In some embodiments, the processor is configured to obtain stable networks of the brain by implying a multi-seed-based correlation analysis by (i) combining the networks from the multiple seed-regions of the brain of the subject within a selected region of interest (ROI) and (ii) weighing the multiple seed-region of the brain of the subject based on a distance from the main seed-region of the brain of the subject.

In some embodiments, the processor is configured to Intraoperative Direct Electrical Stimulation (DES) localization is used for correlation to enable better seed-selection and direct comparison between the DES activation and n brain networks.

In some embodiments, the processor is configured to validate the networks of the brain by comparing the networks of the brain that are obtained based on tasks performed by the subject and correlating with the networks of the brain while the subject is performing a specified task using the interoperative DES localization method, the networks of the brain obtained from task-based are the plurality of networks of the brain obtained from task-based.

In some embodiments, the interoperative DES localization method performs correlation to enable selection of the seed-region of the brain and comparison between the Intraoperative Direct Electrical Stimulation activation and the networks of the brain.

In some embodiments, the networks of the brain is at least one of a primary visual network and sensorimotor network of the brain, a language network, a dorsal default mode network of the brain, a posterior salience network, or a right executive control network of the brain.

In some embodiments, the defined threshold is assigned using a statistical significance for the set of voxels given by the time components of the n-component specified time-series. In some embodiments, composed networks of the brain is converted into at least one of digital imaging and communications in medicine (dicom) format or neuroimaging informatics technology initiative (NIfTI) format in both standard and native space for the subject after optimal thresholding.

The embodiments herein may include a computer program product configured to include a pre-configured set of instructions, which when performed, can result in actions as stated in conjunction with the methods described above. In an example, the pre-configured set of instructions can be stored on a tangible non-transitory computer readable medium or a program storage device. In an example, the tangible non-transitory computer readable medium can be configured to include the set of instructions, which when performed by a device, can cause the device to perform acts similar to the ones described here. Embodiments herein may also include tangible and/or non-transitory computer-readable storage media for carrying or having computer executable instructions or data structures stored thereon.

Generally, program modules utilized herein include routines, programs, components, data structures, objects, and the functions inherent in the design of special-purpose processors, etc. that perform particular tasks or implement particular abstract data types. Computer executable instructions, associated data structures, and program modules represent examples of the program code means for executing steps of the methods disclosed herein. The particular sequence of such executable instructions or associated data structures represents examples of corresponding acts for implementing the functions described in such steps.

The embodiments herein can include both hardware and software elements. The embodiments that are implemented in software include but are not limited to, firmware, resident software, microcode, etc.

A data processing system suitable for storing and/or executing program code will include at least one processor 116 coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

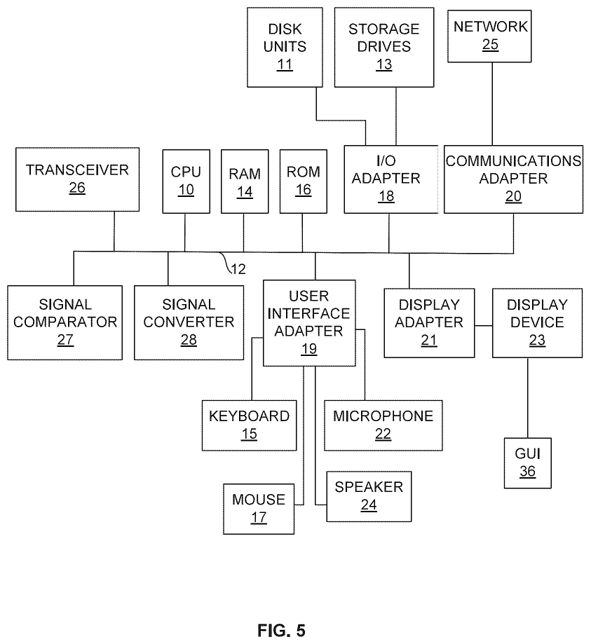

A representative hardware environment for practicing the embodiments herein is depicted in FIG. 5, with reference to FIGS. 1 through 4. This schematic drawing illustrates a hardware configuration of a brain network determining server 108/computer system/computing device in accordance with the embodiments herein. The system includes at least one processing device CPU 10 that may be interconnected via system bus 15 to various devices such as a random-access memory (RAM) 12, read-only memory (ROM) 16, and an input/output (I/O) adapter 18. The I/O adapter 18 can connect to peripheral devices, such as disk units 58 and program storage devices 50 that are readable by the system. The system can read the inventive instructions on the program storage devices 50 and follow these instructions to execute the methodology of the embodiments herein. The system further includes a user interface adapter 22 that connects a keyboard 28, mouse 50, speaker 52, microphone 55, and/or other user interface devices such as a touch screen device (not shown) to the bus 15 to gather user input. Additionally, a communication adapter 20 connects the bus 15 to a data processing network 52, and a display adapter 25 connects the bus 15 to a display device 26, which provides a graphical user interface (GUI) 56 of the output data in accordance with the embodiments herein, or which may be embodied as an output device such as a monitor, printer, or transmitter, for example.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the scope.

I/We claim:

1. A system for determining a plurality of networks of a brain of a subject (102) from a resting state magnetic resonance imaging (MRI) data using a machine learning model for evaluating a plurality of health conditions of the subject (102), the system comprising:
an imaging device (110) that comprises at least one of a camera, or a screen, wherein the imaging device obtains an input data of the subject (102) associated with an expert device (104) that comprises at least one of scan data, wherein the scan data comprises at least one of T1 weighted magnetic resonance imaging (MRI) image, or a resting-state functional MRI image in a predefined format;
a brain network identifying server (108) that acquires the input data of the subject (102) from the imaging device (110), and processes, the input data using the machine learning model (112), wherein the brain network identifying server (108) comprises:
a memory that stores a database;
a processor that is configured to execute the machine learning model (112) and is configured to,
characterized in that,
convert the predefined format of the scan data into an object format file by pre-processing the predefined format of the scan data of the subject (102);
generate, using an independent component analysis method, a four-dimensional (4D) functional connectivity file from the object format file, wherein the 4D functional connectivity file comprises at least one functional connectivity features;
decompose the 4D functional connectivity file into a n-component specified time-series, wherein the n-component specified time-series comprises time components that are independent of each other statistically;
train, using a plurality of data analysis pipelines, the machine learning model (112) by providing a plurality of historical input data of historical subjects and a plurality of historical brain networks associated with the historical subjects as training data to obtain a trained machine learning model;
obtain, using a multi-seed-based correlation analysis, a plurality of networks of the brain of the subject (102) by providing a spatial relationship between a seed-region of the brain of the subject (102) and rest of the object format file of the scan data of the subject (102) when combined;
compose the plurality of networks of the brain of the subject (102) by assigning a defined threshold for a set of voxels of a set of seed-regions of the brain of the subject (102);
determine, using an Intraoperative Direct Electrical Stimulation (DES) localization method and the trained machine learning model, the plurality of networks of the brain by comparing the plurality of composed networks of the brain with a template that defines a network of interest, wherein the brain network identifying server (108) enables the evaluation of a plurality of health conditions of the subject (102) using the plurality of networks that are determined.

2. The system as claimed in claim 1, wherein the processor is configured to pre-process the dicom format of the input data of the subject (102) to obtain a pre-processed dicom format input file by,
discarding a first ten functional time-series volumes of the input data of the subject (102);
interpolating slices of the input data of the subject (102) by acquiring the input data at a single time point by compensating time differences between slice acquisitions of the input data of the subject (102);
correcting a head-movement of the subject (102) that usually occurs during an acquisition of the input data along at least one of X, Y, or Z movement axes and at least one of X, Y, and Z rotation axes;
removing linear or quadratic trends in the functional time-series volumes of the input data of the subject (102);
registering the input data by aligning a functional image of the input data with the reference to a structural image in the input data of the subject (102);
stripping the head of the subject (102) to improve the robustness of the registration to the input data using Montreal Neurological Institute (MNI) normalization;
segmenting the registered input data into a grey matter, a white matter, and a cerebrospinal fluid;
generating a mask for the segmented input data of the brain and scalp regions in both the subject's native space and group mask;
removing noise in a signal induced by the head-movement using regressors, scanner drift using a linear term, and global functional MRI signals from the white matter and the cerebrospinal fluid segments;
filtering noise due to low-frequency drifts and physiological noise using a high-pass filter to remove drifts in neighbouring voxels of the input data; and
removing a small scale that changes among voxels due to an increase in the signal-to-noise ratio of the input data by filtering the high range frequencies from frequency domain.

3. The system as claimed in claim 1, wherein the processor is configured to obtain a plurality of stable networks of the brain by implying a multi-seed-based correlation analysis by (i) combining the plurality of networks from the multiple seed-regions of the brain of the subject (102) within a selected region of interest (ROI) and (ii) weighing the multiple seed-region of the brain of the subject (102) based on a distance from the main seed-region of the brain of the subject (102).

4. The system as claimed in claim 1, wherein the processor is configured to incorporate a dynamic thresholding to increase the threshold of the plurality of networks of the brain to assess an overlap, wherein if the overlap is within an optimal bound, then an optimal thresholding is applied, wherein composed networks of the brain is converted into at least one of the predefined format, or the object format file after optimal thresholding.

5. The system as claimed in claim 1, wherein the processor is configured to validate the plurality of networks of the brain by comparing the plurality of networks of the brain that are obtained based on tasks performed by the subject (102) and correlating with the plurality of networks of the brain while the subject (102) is performing a specified task using the interoperative DES localization method, wherein the plurality of networks of the brain obtained from task-based are the plurality of networks of the brain obtained from task-based.

6. The system as claimed in claim 5, wherein the interoperative DES localization method performs correlation to enable selection of the seed-region of the brain and comparison between the Intraoperative Direct Electrical Stimulation activation and the plurality of networks of the brain.

7. The system as claimed in claim 1, wherein the plurality of networks of the brain is at least one of a primary visual network and sensorimotor network of the brain, a language network, a dorsal default mode network of the brain, a posterior salience network, or a right executive control network of the brain.

8. The system as claimed in claim 1, wherein the predefined format of the scan data comprises at least one of digital imaging and communications in medicine (dicom) format or neuroimaging informatics technology initiative (NIfTI) format, wherein the seed-region of the brain of the subject (102) comprises at least one voxel co-ordinate in the input data, wherein the rest of the object format file of the scan data of the subject (102) is obtained by excluding the seed-region of the brain of the subject (102) from the object format file of the scan data.

9. The system as claimed in claim 1, wherein the defined threshold is assigned using a statistical significance for the set of voxels given by the time components of the n-component specified time-series.

10. A processor-implemented method for determining a plurality of networks of a brain of a subject (102) from a resting state magnetic resonance imaging (MRI) data using a machine learning model for evaluating a plurality of health conditions of the subject (102), the method comprising:
obtaining the input data from an imaging device (110) that comprises at least one of a camera, or a screen, wherein the input data of the subject (102) associated with an expert device (104) comprises at least one of scan data, wherein the scan data comprises at least one of T1 weighted magnetic resonance imaging (MRI) image, or a resting-state functional MRI image, wherein the scan data is in a predefined format;
characterized in that,
converting the predefined format of the scan data into an object format file by pre-processing the predefined format of the scan data of the subject (102);
generating, using an independent component analysis method, a four-dimensional (4D) functional connectivity file from the object format file, wherein the 4D functional connectivity file comprises at least one functional connectivity features;
decomposing the 4D functional connectivity file into a n-component specified time-series, wherein the n-component specified time-series comprises time components that are independent of each other statistically;
training, using a plurality of data analysis pipelines, the machine learning model by providing a plurality of historical input data of historical subjects and a plurality of historical brain networks associated with the historical subjects as training data to obtain a trained machine learning model;
obtaining, using a multi-seed-based correlation analysis, a plurality of networks of the brain of the subject (102) by providing a spatial relationship between a seed-region of the brain of the subject (102) and rest of the object format file of the scan data of the subject (102) when combined;
composing the plurality of networks of the brain of the subject (102) by assigning a defined threshold for a set of voxels of a set of seed-regions of the brain of the subject (102);
determining, using an Intraoperative Direct Electrical Stimulation (DES) localization method and the trained machine learning model, the plurality of networks of the brain by comparing the plurality of composed networks of the brain with a template that defines a network of interest, wherein the brain network identifying server (108) enables the evaluation of a plurality of health conditions of the subject (102) using the plurality of networks that are determined.
